(19) United States
(12) Patent Application Publication
(10) Pub. No.: US 2022/0031763 A1
(43) Pub. Date: Feb. 3, 2022

(54) ANTI-ACHING SUPPLEMENT

(71) Applicant: NGOZICHUKWU AKUBUIKE, SAINT PAUL, MN (US)

(72) Inventor: NGOZICHUKWU AKUBUIKE, SAINT PAUL, MN (US)

(21) Appl. No.: 16/940,741

(22) Filed: Jul. 28, 2020

(51) Int. Cl. A61K 35/644; A61K 36/185; A61K 36/9066; A61K 36/67; A61K 47/44; A61K 9/06; A61K 9/107; A61K 36/9068; A61K 36/54; A61P 29/00

(52) U.S. Cl. CPC A61K 35/644; A61K 36/185; A61K 36/9066; A61K 36/67; A61P 29/00; A61K 9/06; A61K 9/107; A61K 36/9068; A61K 36/54; A61K 47/44

(57) ABSTRACT

A composition of phyto-medicaments comprising essentially of Piper guineense, turmeric and Moringa oleifera for the treatment necessary to alleviate ailments associated therewith.

### BACKGROUND

People who suffer from chronic pains, nagging health issues such as stress and depression, addictions, inflammation and matters of immune health can attest to the challenges and desperation in getting or obtaining treatment for their ailments without the rigor of suffering with adverse effects of the medicaments that they have to take. It is also an issue of concern that almost all the medicament taken or consumed by these patients present yet again foreign material or abstracts into them. One can presently imagine taking opioids for pain and suffering from diarrhea as an outcome, or becoming addicted to the opioids, just as examples.

In other instances, nutraceuticals and other pharmaceuticals may present medicaments that come with issues of potency and sourcing. Potency, in that nutraceuticals may come from sources that may affect the efficacy of the medicament and quality of the raw materials used. Biologics offer some solutions to sufferers and there is recognition that biologics may provide challenges because of the process and the additives that make them usable for their intended purpose or purposes.

Drugs, and other non-homeopathic formulations or similarly fabricated medicines used to treat issues of anti-inflammation, agues or weight management, among others are typically tied to some adverse effects of such treatment. It is thus reasonable to imagine patients who would rather suffer than take medication that would cause them more challenges than those they already know and manage.

From the foregoing and more, it will be appreciated that what is needed in the art is a formulation that provides some relief to sufferers of stomach churns, body aches and pains and related challenges that is devoid of the attendant adverse effects.

Such system is disclosed and claimed herein.

### SUMMARY

In one embodiment, the present invention provides a combination of phyto-medicaments comprising essentially of Piper guineense, turmeric and Moringa oleifera for the treatment necessary to alleviate ailments associated therewith.

In another embodiment of the present invention, the phyto-medicament composition consists of Piper guineense, Moringa oleifera, turmeric and ginger formulated for the purpose of providing relief to patients of aches and ailments of the related to inflammation of the nerves.

The composition of the present invention may preferably include the aforesaid phyto-medicament with additives, such as aromatic olive oils, Cinnamomum verum, honey and minerals.

### DETAILED DESCRIPTION

The present invention provides a composition of phyto-medicaments, uniquely formulated for the relief of body aches and inflammation challenges including, and not limited to those useful for the treatment of body aches, agues, inflammations, high blood pressure and weight management. Such challenges include stomach churns and similar aches that affect a large population of folks, in some cases, after food intake or consumption of medications

The present invention comprises aromatic olive oil, Piper guineense, turmeric and Moringa oleifera and, in some optional circumstance, ginger, cinnamon and honey as an adjuvant for taste enhancement. Preferably, the turmeric usable in the instant disclosure is derived from the plant or root form of Curcuma longa and not previously processed into a powder. The transition into powdery texture of the turmeric usable in the present invention is preferably done by milling or grinding such raw turmeric into an emulsion, in the form of a paste or powder suitable to be formed into a paste by combination or mixture of some inactive or inert solvent to form a paste or such emulsion or composition. The milling or grinding of the turmeric root may be accomplished via known industrial process that may include drying the root to a suitable condition for pounding in a mortar with the use of a pestle, grating or chopping the roots into fine pieces for further blending or pureeing in a blender.

Commercial equipment such as Nutribullet blenders have been operably usable to produce the emulsions or pastes as needed for the production of the phyto-medicament of the present invention. Preferable amount usable in the present invention comprises between about 1 (8 ounces) and 4 cups (32 ounces), more preferably between about 1.5 and 3 cups and most preferably between about 1.75 and 2.5 cups per dose or formulation (noting that 1 cup is about 8 ounces).

The present invention further comprises Piper guineense, said Piper guineense preferably being available as seeds for use in the instant formulation. Given the prevalence of Piper guineense in many parts of the world, preferable origin of the usable form of Piper guineense is a tropically and organically grown variety from West Africa. The version of the seeds usable in the present invention are preferably dry to between about 10 to 20 percent moisture content. In some instances, wherein the Piper guineense is exceedingly dry, a measure of purified water may be usable to attain a desired moisture content. Further processing of the Piper guineense into ground powder may including pounding in a mortar and pestle of blending using available blenders, preferably Nutribullet brand available from Nutribullet Company of Los Angeles, Calif. Useful amount for formulation of the present invention include preferably between about 1 and 3 teaspoonfuls; more preferably between about 1 and 2.5 teaspoonfuls and most preferably between about 1 (0.17 ounces) and 2 teaspoonfuls (about 0.33 ounces) of Piper guineense per formulation.

The Moringa oleifera usable in the instant disclosure may be in the form of leaves. Moringa leaves usable in the present invention are preferably collected and air-dried. The dried leaves are preferably blended prior to further blending or combination with other components of the present invention. Moringa oleifera leaves are most usable as normal green leaves and prior to any discoloration which my signify loss of chlorophyll or diminution in quality. Ground Moringa oleifera usable in about one cup (8 ounces) per formulation according to the present invention.

The present invention optionally includes ginger (Zingiber officinale) in root form. Ginger, usable in the present invention is preferably available in root form. Generally, ginger is in component, at least about 70% water and prepared when washed, chopped and ground or blended for use in the present invention. Preparation of the ginger into usable quality or form may preferably be accomplished using blenders such as Nutribullet blenders to operably produce the emulsions or pastes as needed for the production of the phyto-medicament of the present invention. An amount usable for the present invention may be between about 1 measured cup (8 oz.) and about 1.5 measured cups (12 oz.) of ginger.

The present invention may also include cinnamon (Cinnamomum verum) in the form of Ceylon Cinnamon. Ceylon Cinnamon is preferentially referred to as true cinnamon, generally sweet and more delicate in flavor than cassia cinnamon. Ceylon cinnamon, typically in stick forms are usable in the present invention. As in other components usable in the present invention, the Ceylon Cinnamon is ground into a powder form. About 2 sticks of Ceylon Cinnamon provides about 1 teaspoonful of ground cinnamon, in quantity suitable to use in the present invention.

Further optional adjuvants in the present invention may include aromatic olive oil, which preferably is prepared from ripe olive fruits for a milder and more buttery oil. Olive oil usable in this preparation may be limited in quantity to provide adequate texture and consistency for the formulation of a paste. An amount of less than about a teaspoonful of aromatic olive oil may be adequate to prepare the formulation needed in the present invention.

The present invention may also include water (preferably purified) and honey to suitable taste, texture and composition.

The present invention may also useful for the prevention or management of weight, bowel movements, fibromyalgia and more. While these are subject to future testimonials, user experience and results support efficacies in these areas. In use, the present invention may be blended, and imbibed in 2 tablespoonfuls (0.33 oz) per 8 ounces of hot water.

There may be other non-essential adjuvants that may be included as production demands for the preservation, availability, appeal, taste or other such attributes without affecting the efficacy or functionality of the composition of the present invention.

### Example I

A composition comprising about 2 cups of Turmeric, 1 cup of Moringa oleifera, 1 cup of ginger, 2 teaspoonfuls of Piper guineense, 1 teaspoonful of Ceylon Cinnamon, less than 1 teaspoonful of olive oil; all blended to a paste. The paste was thereafter used as a base formulation and 2 tablespoonfuls stirred in an 8 ounce of hot water. Same dosage was provided for relief from high blood pressure on a regular bases of each morning and night. A person who presented with high-blood pressure saw relief after 2 weeks of use of the present invention

### Example II

A middle-aged female, who has had chronic waist pain for over 10 years and challenges from fibromyalgia took the formulation of Example I above. After 4 weeks of consistent use, the fibromyalgia and waist pain were mitigated and said ailments have not returned.

Having thus described the preferred embodiments of the present invention, those of skill in the art will readily appreciate that the teachings found herein may be applied to yet other embodiments within the scope of the claims hereto attached. The complete disclosure of all patents, patent documents, and publications are incorporated herein by reference as if individually incorporated.

What is claimed:

1. An anti-aching composition for relieving body aches in a mammal comprising:
between about 8 and 20 ounces of turmeric,
between about 4 and 12 ounces of Moringa oleifera,
between about I and 2 teaspoonfuls of Piper guineense, and
about 1 teaspoonful of olive oil.
wherein said composition was useful for relief of aches in a mammal.

2. The composition of claim 1, wherein the formulation is available as a paste or emulsion.

3. The composition of claim 1, further comprising between about 4 and 12 ounces of ginger.

4. The composition of claim 1, further comprising about 1 teaspoonful of Ceylon Cinnamon.

5. The composition of claim 1, wherein adjuvants selected from the group consisting ginger, Ceylon Cinnamon, honey or a combination thereof.

6. The composition of claim 1, wherein water is added to texture.
